SEQUENCE LISTING (8 sequences)

SEQ ID NO: 1 - DNA, 27 bp, Artificial sequence
  Primer bind
gttgacgttaccyacagaagaagcacc

SEQ ID NO: 2 - DNA, 20 bp, Artificial sequence
  Primer bind
gggctttcacacccaactta

SEQ ID NO: 3 - DNA, 23 bp, Artificial sequence
  Primer bind
aaaccgcctacgcgccctttacg

SEQ ID NO: 4 - DNA, 19 bp, Artificial sequence
  Primer bind
cgggaggaaaagcatcggg

SEQ ID NO: 5 - DNA, 19 bp, Artificial sequence
  Primer bind
tccgaccgcctacatgcgc

SEQ ID NO: 6 - DNA, 19 bp, Artificial sequence
  Primer bind
ggagraaagcaggggatcg

SEQ ID NO: 7 - DNA, 19 bp, Artificial sequence
  Primer bind
ggaggaaagtaggggatcg

SEQ ID NO: 8 - DNA, 22 bp, Artificial sequence
  Primer bind
cgtcctctcagaccarctactg

(19) United States
(12) Patent Application Publication
(10) Pub. No.: US 2024/0166988 A1
(43) Pub. Date: May 23, 2024

(54) NITRIFYING BACTERIA FORMULATION

(71) Applicant: JFR CO., LTD., Tokyo (JP)

(72) Inventors: Yuko SASAGAWA, Tokyo (JP); Akira FUJINO, Tokyo (JP); Hiroshi MIYAZAKI, Tokyo (JP)

(73) Assignee: JFR CO., LTD., Tokyo (JP)

(21) Appl. No.: 18/551,272

(22) PCT Filed: Mar. 29, 2022
(86) PCT No.: PCT/JP2022/015372

§ 371 Date: Sep. 19, 2023

(30) Foreign Application Priority Data
Mar. 31, 2021 (JP) 2021-062128

(51) Int. Cl. C12N 1/20; C12N 11/00

(52) U.S. Cl. CPC C12N 1/20; C12N 11/00; C12N 2500/05

(57) ABSTRACT

A nitrifying bacteria formulation that can cause a sufficient nitrification reaction even in a high ionic concentration (salt concentration), and efficiently collect high-concentration nitrate-form nitrogen from ammonia produced in animal husbandry facilities, composting facilities, sewage treatment plants, etc.; and a method for culturing bacteria to be contained in the nitrifying bacteria formulation. A nitrifying bacteria formulation includes: ammonia-oxidizing bacteria and nitrite-oxidizing bacteria, wherein the ammonia-oxidizing bacteria include bacteria belonging to the Nitrosococcus, the proportion of the number of the bacteria belonging to the Nitrosococcus with respect to the total number of bacteria in the nitrifying bacteria formulation is at least 0.1%, and the proportion of the number of the nitrite-oxidizing bacteria with respect to the total number of bacteria in the nitrifying bacteria formulation is at least 0.1%. A method for culturing bacteria to be contained in the nitrifying bacteria formulation may also be provided.

### TECHNICAL FIELD

The present invention relates to a nitrifying bacteria formulation and a method for culturing bacteria contained therein.

### BACKGROUND

Conventionally, offensive odors containing ammonia as a main component have been produced in e.g. livestock farms, composting plants and night soil treatment plants, and problems of complaints from neighborhood against the offensive odors and health damages of workers in the fields have frequently occurred. It has been therefore required to treat the produced ammonia by deodorization equipment, and various deodorization methods have been suggested. Biological deodorization using nitrifying bacteria, for example, has been widely used, in which nitrifying bacteria decompose ammonia by oxidizing it to nitrous acid and further to nitric acid (e.g. Japanese Unexamined Patent Application, Publication No. 2008-516603 and Japanese Unexamined Patent Application, Publication No. 2019-202244).

### DISCLOSURE OF THE INVENTION

The conventional technique using nitrifying bacteria, however, has not been able to sufficiently treat ammonia particularly under a treatment condition having a high ion concentration (salt concentration) in some cases. In the case of bacteria of the genus Nitrosomonas as described in Patent Document 1, for example, the activity thereof can be significantly reduced under a high ion concentration.

The present invention has been made in view of the above actual circumstances, and an object thereof is to provide a nitrifying bacteria formulation that is capable of allowing a sufficient nitrification reaction to proceed under various treatment conditions, particularly even under a treatment condition having a high ion concentration (salt concentration) and recovering nitrate nitrogen from ammonia, and a method for culturing bacteria contained therein.

As a result of diligent consideration and research to solve the above problem, the present inventors have found that by a nitrifying bacteria formulation containing specific bacterial species as ammonia-oxidizing bacteria (AOB) and containing the AOB and nitrite-oxidizing bacteria (NOB) in a specific rate or more, the nitrifying activity thereof is retained and nitrogen in ammonia can be recovered as high-concentration nitrate ion even under a treatment condition having a high ion concentration (salt concentration), thereby completing the present invention.

That is, the present invention provides the following:

(1) A nitrifying bacteria formulation, containing ammonia-oxidizing bacteria and nitrite-oxidizing bacteria,
the ammonia-oxidizing bacteria containing bacteria belonging to the genus Nitrosococcus,
the proportion of the number of the bacteria belonging to the genus Nitrosococcus with respect to the total number of bacteria in the nitrifying bacteria formulation being 0.1% or more,
the proportion of the number of the nitrite-oxidizing bacteria with respect to the total number of bacteria in the nitrifying bacteria formulation being 0.1% or more.
   (2) The nitrifying bacteria formulation according to aspect (1), wherein the proportion of the number of the bacteria belonging to the genus Nitrosococcus with respect to the total number of bacteria in the nitrifying bacteria formulation is 1.0% or more.
   (3) The nitrifying bacteria formulation according to aspect (1), wherein the proportion of the number of the bacteria belonging to the genus Nitrosococcus with respect to the total number of bacteria in the nitrifying bacteria formulation is 7.0% or more.
   (4) The nitrifying bacteria formulation according to any one of aspects (1) to (3), wherein the nitrite-oxidizing bacteria contain bacteria belonging to the genus Nitrococcus.
   (5) The nitrifying bacteria formulation according to any one of aspects (1) to (4), wherein the nitrifying bacteria formulation includes a liquid culture of the ammonia-oxidizing bacteria and the nitrite-oxidizing bacteria.
   (6) The nitrifying bacteria formulation according to any one of aspects (1) to (4), wherein the nitrifying bacteria formulation includes pellets or freeze-dried product of the ammonia-oxidizing bacteria and the nitrite-oxidizing bacteria.
   (7) The nitrifying bacteria formulation according to any one of aspects (1) to (4), wherein the nitrifying bacteria formulation includes a nitrifying bacteria-carrying body in which the ammonia-oxidizing bacteria and the nitrite-oxidizing bacteria are supported on a carrier.
   (8) A method for culturing the ammonia-oxidizing bacteria and the nitrite-oxidizing bacteria contained in the nitrifying bacteria formulation according to any one of aspects (1) to (4), the method including,
bringing seed bacteria containing the bacteria belonging to the genus Nitrosococcus and the nitrite-oxidizing bacteria into contact with an ammonia component in a medium in a wet state under an oxygen atmosphere with a salt concentration maintained at 0.5 mol/L or more and 1 mol/L or less.
   (9) The culturing method according to (8), wherein the medium has a magnesium concentration of 0.5 mmol/L or more and a phosphorus concentration of 0.5 mmol/L or more.

According to the present invention, a nitrifying bacteria formulation is provided that is capable of performing a sufficient nitrification reaction under various treatment conditions, particularly even under a treatment condition having a high ion concentration (salt concentration) and recovering nitrate nitrogen from ammonia, and a method for culturing bacteria contained therein.

### BRIEF DESCRIPTION OF THE DRAWINGS

FIG. 1 is diagram which schematically shows a structure of a nitrogen recovery device used in Example 4.

### DETAILED DESCRIPTION OF EMBODIMENTS

The present invention will now be described in detail by way of embodiments.

### <Nitrifying Bacteria Formulation>

The nitrifying bacteria formulation in the present invention is a nitrifying bacteria formulation, containing ammonia-oxidizing bacteria and nitrite-oxidizing bacteria, wherein the ammonia-oxidizing bacteria contain bacteria belonging to the genus Nitrosococcus, the proportion of the number of the bacteria belonging to the genus Nitrosococcus with respect to the total number of bacteria in the nitrifying bacteria formulation is 0.1% or more, and the proportion of the number of the nitrite-oxidizing bacteria with respect to the total number of bacteria in the nitrifying bacteria formulation is 0.1% or more.

The bacteria belonging to the genus Nitrosococcus have been known to show an ammonia nitrifying activity even under a condition having a high NaCl concentration such as sea water. This time, however, the present inventors have found new knowledge that the bacteria have a good ammonia oxidizing activity even under a treatment condition having high-concentration nitrates, for example, even at a high ion concentration such as a nitrate ion concentration of 3 w/v % (30,000 mg/L, about 0.5 mol/L) or more. Therefore, according to the present invention, an ammonia oxidizing reaction proceeds well even under a treatment condition having a high ion concentration by the existence of the bacteria belonging to the genus Nitrosococcus. A sufficient amount of resulting nitrous acid is oxidized to nitrate by the action of the nitrite-oxidizing bacteria. Therefore, according to the nitrifying bacteria formulation in the present invention, nitrate ion is accumulated at a high concentration (e.g. 30,000 mg/L or more) in water to be treated, and nitrogen in ammonia can be also efficiently recovered.

The ammonia-oxidizing bacteria and the nitrite-oxidizing bacteria may be now referred to as “AOB” and “NOB” respectively.

As a result of consideration, the present inventors have found unexpected knowledge that ammonia can be nitrified even under various conditions such as a high ion concentration by adjusting the proportion of the bacteria belonging to the genus Nitrosococcus to 0.1% or more in the presence of the nitrite-oxidizing bacteria. As shown also in Examples below, according to the nitrifying bacteria formulation in the present invention, even if a subject to be treated has a high ion concentration like sea water (average salt concentration: more than 3 w/v %), ammonia can be sufficiently nitrified. Therefore, a preferred subject to be treated for the nitrifying bacteria formulation in the present invention is water to be treated having a high ion concentration (e.g. sea water, liquid waste from plants, liquid waste by fermentation, and high-concentration ammonia water generated during the deodorization of an ammonia gas).

The “high ion concentration” in the present invention encompasses ion concentrations such as 3 w/v % (30,000 mg/L) or more, particularly 5 w/v % (50,000 mg/L) or more. The “ion” in the present invention varies depending on subjects to be treated and operating conditions such as chemicals used for pH adjustment, and encompasses any ion and particularly indicates nitrate ion and ions forming nitrates (such as sodium nitrate, calcium nitrate, potassium nitrate and magnesium nitrate).

The “high ion concentration” in the present invention can be, for example, 0.5 mol/L (M) or more, particularly 1 mol/L or more in terms of nitrate ion concentration. It should be noted that because nitrate and the like is ionized in water, the total ion concentration including cations is a value twice or more a nitrate ion concentration and a salt concentration. The present invention, however, focuses particularly on e.g. nitrate ion, and thus the molar concentration of ion is provided by the anion concentration or salt concentration.

In the present invention, the ion concentration in a subject to be treated can be measured by e.g. zinc reduction-naphthylethylenediamine absorptiometry, ion chromatography and colorimetry. A pretreatment, for example, may be separately performed depending on the state of a subject to be measured (for example, when a large amount of nitrite ion is included in a liquid).

The structure of the nitrifying bacteria formulation in the present invention will now be described in detail.

### (Ammonia-Oxidizing Bacteria)

The ammonia-oxidizing bacteria (AOB) are bacteria which oxidize ammonia to nitrous acid. The nitrifying bacteria formulation in the present invention contains at least the bacteria belonging to the genus Nitrosococcus as AOB.

### (The Genus Nitrosococcus)

As the bacteria belonging to the genus Nitrosococcus, those which are conventionally known can be used. Examples thereof include Nitrosococcus mobilis, Nitrosococcus nitrosus, Nitrosococcus oceanus (Nitrosococcus oceani), Nitrosococcus watosonii, Nitrosococcus halophilus and the like. A plurality of the bacteria belonging to the genus Nitrosococcus can be also used in combination.

In the nitrifying bacteria formulation in the present invention, the proportion of the number of the bacteria belonging to the genus Nitrosococcus with respect to the total number of bacteria in the nitrifying bacteria formulation is 0.1% or more, preferably 1.0% or more, more preferably 3.0% or more, further preferably 7.0% or more and particularly preferably 10.0% or more.

The proportion of the number of the bacteria belonging to the genus Nitrosococcus in the nitrifying bacteria formulation can be determined as the proportion of the number of bacteria of e.g. Nitrosococcus with respect to the total number of bacteria by analyzing 16S rRNA gene using next generation sequencing or real-time PCR.

It should be noted that the number of the bacteria belonging to the genus Nitrosococcus described herein is the proportion of the number of bacteria in the nitrifying bacteria formulation. The number of the bacteria belonging to the genus Nitrosococcus with respect to the total number of bacteria in a subject to be treated can be less than 0.1% at a time point when the nitrifying bacteria formulation is brought into contact with the subject to be treated (for example, when the nitrifying bacteria formulation is put in a microbial decomposition tank in a treatment device). Even in such a case, however, the bacteria belonging to the genus Nitrosococcus grow over time, and can treat ammonia. However, as described below, in order to recover nitrate ion quickly, the proportion of the number of the bacteria belonging to the genus Nitrosococcus with respect to the total number of bacteria in a subject to be treated, for example, in a microbial decomposition tank is preferably 0.1% or more from the start point of nitrogen recovery. Therefore, the proportion of the number of the bacteria belonging to the genus Nitrosococcus with respect to the total number of bacteria in the nitrifying bacteria formulation is, for example, preferably 1.0% or more, further preferably 3.0% or more, especially preferably 7.0% or more and particularly preferably 10.0% or more.

The nitrifying bacteria formulation in the present invention may or may not further include one or more kinds of AOB other than the bacteria belonging to the genus Nitrosococcus. Examples of AOB other than the bacteria belonging to the genus Nitrosococcus include various bacteria such as bacteria belonging to the genus Nitrosomonas, the genus Nitrosospira, the genus Nitrosolobus, the genus Brevibacillus and the genus Xanthomonas.

As described below, the nitrifying bacteria formulation in the present invention can be prepared, for example, by using activated sludge as seed bacteria, and in such activated sludge, various AOB including the bacteria of the genus Nitrosomonas frequently exist. Therefore, AOB described above may coexist in the nitrifying bacteria formulation in the present invention. The nitrifying bacteria formulation in the present invention may contain AOB other than the bacteria of the genus Nitrosococcus, for example, bacteria of the genus Nitrosomonas at about 1 to 70%, particularly about 4 to 30%. Although the mechanism is not known, when the bacteria belonging to the genus Nitrosococcus are contained with other AOB, the nitrification ability thereof can be promoted by a synergistic effect.

### (Nitrite-Oxidizing Bacteria)

The nitrite-oxidizing bacteria (NOB) are bacteria which oxidize nitrous acid to nitric acid.

As NOB, those which are conventionally known can be used. Examples thereof include, but not limited to, bacteria of the genus Nitrococcus; bacteria of the genus Nitrobacter such as Nitrobacter winogradskyi, Nitrobacter alkalicus, Nitrobacter vulgaris and Nitrobacter hamburgensis; bacteria belonging to the genus Nitrospira such as Nitrospira marina and Nitrospira moscoviensis, and the like. A plurality of NOB can be used in combination.

In the nitrifying bacteria formulation in the present invention, the proportion of the number of NOB with respect to the total number of bacteria in the nitrifying bacteria formulation is 0.1% or more. When the rate of NOB to the total number of bacteria is 0.1% or more, the oxidation of nitrous acid sufficiently proceeds even under an environment having a high ion concentration. In the nitrifying bacteria formulation in the present invention, as the rate of NOB, the proportion of the number of NOB with respect to the total number of bacteria in the nitrifying bacteria formulation is preferably 0.5% or more, more preferably 1.0% or more, further preferably 1.5% or more and particularly preferably 2.0% or more.

The proportion of the number of NOB in the nitrifying bacteria formulation can be determined as the proportion of the number of bacteria of e.g. Nitrococcus with respect to the total number of bacteria by analyzing 16S rRNA gene using next generation sequencing or real-time PCR.

It should be noted that the number of NOB described herein is the proportion of the number of bacteria in the nitrifying bacteria formulation. The number of NOB with respect to the total number of bacteria in a subject to be treated can be less than 0.1% at a time point when the nitrifying bacteria formulation is brought into contact with the subject to be treated (for example, when the nitrifying bacteria formulation is put in a microbial decomposition tank in a treatment device). Even in such case, however, NOB grow over time, and can allow nitrification to proceed. However, as described below, in order to recover nitrate ion quickly, the proportion of the number of NOB with respect to the total number of bacteria in a subject to be treated, for example, in a microbial decomposition tank is preferably 0.1% or more from the start point of nitrogen recovery. Therefore, the proportion of the number of NOB with respect to the total number of bacteria in the nitrifying bacteria formulation is preferably 0.5% or more, further preferably 1.0% or more, yet further preferably 1.5% or more and particularly preferably 2.0% or more.

### (The Genus Nitrococcus)

From the viewpoint of having a particularly good activity even under a treatment condition having a high ion concentration, the nitrifying bacteria formulation in the present invention preferably contain the bacteria belonging to the genus Nitrococcus as NOB. The bacteria belonging to the genus Nitrococcus can grow and function at a low salt concentration or a high salt concentration. Examples of the bacteria belonging to the genus Nitrococcus include Nitrococcus mobilis and the like.

### (Preparation of Nitrifying Bacteria Formulation)

The nitrifying bacteria formulation in the present invention can be prepared, for example, using activated sludge as seed bacteria. For example, it can be prepared by a culturing method, including bringing seed bacteria containing the bacteria belonging to the genus Nitrosococcus and the nitrite-oxidizing bacteria into contact with an ammonia component in a medium in a wet state under an oxygen atmosphere with the ion concentration maintained at about 0.5 mol/L or more and 1 mol/L or less. As described above, AOB such as the bacteria belonging to the genus Nitrosococcus and NOB such as the bacteria belonging to the genus Nitrococcus can grow even in a high salt concentration, and thus it can be produced by culturing and screening under such environment having a high ion concentration.

As the seed bacteria, optional seed bacteria can be used as long as AOB containing the bacteria belonging to the genus Nitrosococcus and NOB are included herein.

The bacteria belonging to the genus Nitrosococcus and the nitrite-oxidizing bacteria usually require time to grow, and thus in order to increase the proportion of these bacteria early in the preparation of the nitrifying bacteria formulation, the proportion of the number of the bacteria belonging to the genus Nitrosococcus with respect to the total number of bacteria in seed bacteria (hereinafter may be described as “proportion of existing Nitrosococcus”) is 0.1% or more, more preferably 1.0% or more, further preferably 3.0% or more, yet further preferably 7.0% or more and particularly preferably 10.0% or more, and also the proportion of the number of NOB with respect to the total number of bacteria in seed bacteria (hereinafter, may be described as “proportion of existing NOB”) is 0.1% or more, more preferably 0.5% or more, further preferably 1.0% or more and particularly preferably 2.0% or more.

The upper limits of the proportion of existing Nitrosococcus and the proportion of existing NOB in seed bacteria are not particularly limited, and from the viewpoint that among the nitrification reaction, the ammonia oxidizing reaction does not easily proceed compared to the nitrite-oxidizing reaction, it is preferred that both the proportions be in the same degree or the proportion of existing Nitrosococcus be higher. For example, both the proportion of existing Nitrosococcus and the proportion of existing NOB in seed bacteria with respect to all microorganisms in the seed bacteria are preferably up to 30 to 50%. For example, the proportion of existing Nitrosococcus and the proportion of existing NOB in seed bacteria are preferably Nitrosococcus:NOB=2:1 to 30:1 and more preferably 3:1 to 20:1. It is also preferred that the proportion of existing Nitrosococcus and the proportion of existing NOB in the nitrifying bacteria formulation have the same ratio.

The proportion of existing respective bacteria in seed bacteria is determined, for example, by the above 16S rRNA gene bacterial flora analysis.

When using activated sludge as seed bacteria, e.g. activated sludge derived from swine waste water, treated by an activated sludge method in swine waste water treatment facilities may be used.

The activated sludge may be subjected to the so-called preculture treatment. It should be noted that the “preculture” described herein is a pretreatment in which an ammonia component is added to activated sludge as needed and the activated sludge is then placed, for example, in an aerobic condition at normal temperature to grow or activate bacteria before culturing bacteria by the contact of an ammonia component while adjusting the ion concentration as described above, i.e. main culture. In particular, for example, when sludge after being collected is stored under refrigeration, the state of bacteria can be restored by the preculture. For example, sludge such as activated sludge from swine waste water treatment facilities can be used for the main culture after being pretreated by aerobically culturing at 15 to 40° C., particularly 20 to 35° C. over a day to several weeks.

In order to prepare the nitrifying bacteria formulation in the present invention, ammonia is preferably supplied at the time of culturing as described above. The bacteria belonging to the genus Nitrosococcus grow using the supplied ammonia as a nutrient source, and thus the rate thereof can be increased. In addition, NOB and the bacteria belonging to the genus Nitrococcus grow using nitrous acid produced by Nitrosococcus as a nutrient source, and the composition of bacteria in the nitrifying bacteria formulation in the present invention can be obtained.

The pH at the time of culturing is preferably adjusted to about 5 to 8 and particularly about 6.5 to 7.5. In the culturing of the bacteria belonging to the genus Nitrosococcus and NOB, as the nitrification reaction proceeds, the pH of a culture fluid is reduced, and thus pH is preferably adjusted by adding an alkali. Also from such viewpoint, it is desired that ammonia be supplied at the time of culturing.

In addition, in the preparation of the nitrifying bacteria formulation in the present invention, culturing is preferably performed in a condition having a high ion concentration, for example, a nitrate ion concentration of 30,000 mg/L or more, particularly 30,000 to 50,000 mg/L, or a salt concentration of 0.5 M or more. As described above, Nitrosococcus is resistant to a condition having a high ion (salt) concentration, and thus tends to grow even under an environment in which other AOB are killed. Therefore, a nitrifying bacteria formulation in which the proportion of the number of particularly the bacteria belonging to the genus Nitrosococcus among AOB is high can be prepared by culturing in a condition having a high ion concentration. However, when the salt concentration is above 1 M, the nitrifying activity may be slightly reduced, and thus, for example, dilution is performed at a time point when the salt concentration is above 1 M, and culturing may be allowed to proceed at a salt concentration of about 0.5 to 1 M.

In addition, as described below, the proportions of the numbers of the bacteria belonging to the genus Nitrosococcus and NOB can be increased by culturing in a condition having a magnesium concentration of 0.5 mM (mmol/L) or more and especially about 0.6 to 1.0 mM, and a phosphorus concentration of 0.5 mM or more and especially about 0.6 to 1.0 mM. Consequently, a nitrifying bacteria formulation in which the proportion of each number of the bacteria belonging to the genus Nitrosococcus and NOB with respect to the total number of bacteria in the nitrifying bacteria formulation is 0.1% or more is obtained. In particular, a nitrifying bacteria formulation in which the proportion of the number of the bacteria belonging to the genus Nitrosococcus is preferably 1.0% or more, more preferably 3.0% or more, further preferably 7.0% or more and further suitably 10.0% or more can be prepared.

The ambient phosphorus concentration is reduced by the growth of microorganisms such as the bacteria belonging to the genus Nitrosococcus, and thus the phosphoric acid concentration of a medium is measured on a regular basis, and phosphate ion is preferably added as needed at the time of culturing. A phosphorus salt such as K₂HPO₄, for example, may be added with a magnesium salt such as MgSO₄·7H₂O. It is preferred that culturing be performed under a condition having a magnesium concentration of 0.5 mmol/L or more and a phosphorus concentration of 0.5 mmol/L or more in a medium.

From the viewpoint of screening in a high ion concentration environment, the nitrifying bacteria formulation in the present invention, which has been already used for the nitrification treatment and exposed to a treatment liquid having a high ion concentration, may be used as seed bacteria. In addition, a nitrifying bacteria formulation after the nitrification treatment in a small-scale device, for example, is directly put on a large-scall nitrification treatment device, and can be also used as a new nitrifying bacteria formulation.

### (Nitrifying Bacteria Formulation)

The nitrifying bacteria formulation in the present invention may be in any form as long as it contains the bacteria belonging to the genus Nitrosococcus and NOB in equal to or higher than predetermined proportions of the numbers of bacteria thereof, and it may be, for example, in a liquid form in which these bacteria are dispersed in water. Examples thereof include, but not limited to, a nitrifying bacteria formulation including a liquid culture of AOB (ammonia-oxidizing bacteria) containing the bacteria belonging to the genus Nitrosococcus and NOB (nitrite-oxidizing bacteria), and the like. Alternatively, for example, the nitrifying bacteria formulation in a liquid form may be separated by an operation such as centrifugation or filtration, followed by producing a solid such as pellets. It may be also freeze-dried product, which are e.g. powders obtained by freeze-drying a liquid culture or pellets. These bacteria can be also supported on a carrier. The present invention also encompasses a nitrifying bacteria formulation including pellets of AOB containing the bacteria belonging to the genus Nitrosococcus and NOB, and a nitrifying bacteria formulation including a nitrifying bacteria-carrying body in which AOB containing the bacteria belonging to the genus Nitrosococcus and NOB are supported on a carrier.

In light of ease of handling and ammonia treatment efficiency, the nitrifying bacteria formulation preferably has a form of the nitrifying bacteria-carrying body in which nitrifying bacteria including AOB (ammonia-oxidizing bacteria) containing the bacteria belonging to the genus Nitrosococcus and NOB (nitrite-oxidizing bacteria) are supported on a carrier. The ammonia nitrification by the bacteria belonging to the genus Nitrosococcus and NOB is an aerobic reaction, and thus in the case of a nitrifying bacteria formulation in which bacteria are supported particularly to a porous or fibrous carrier, oxygen is sufficiently supplied and the reaction easily proceeds. The amount of water which can be retained in the carrier described above (amount of water absorption) is high, and the amount of an ammonia component which can be absorbed in the water is also high, and thus ammonia nitrification can be promoted.

### (Carrier)

The carrier to which the bacteria belonging to the genus Nitrosococcus and NOB are supported (nitrifying bacteria-carrying body) is not particularly limited, and a material which has a large surface area and is not easily corrupted is preferably used. Examples thereof include, but not limited to, porous bodies and/or inorganic fibrous bodies such as zeolite, a porous glass such as a foam glass, perlite, diatomite, pumice, Oya stone, aggregates such as calcium carbonate particles and barium sulfate particles, silica gel, rock wool, glass wool, a carbon fiber, and the like. Considering ease of handling and costs, zeolite, a porous glass, glass wool, etc. particularly a porous material such as a foam glass are preferably used.

As long as the carrier is a porous body, particularly, having an average particle diameter classified into a desired range such as above 1 mm and 1.4 mm or less, above 1.4 mm and 2 mm or less, above 2 mm and 2.8 mm or less, above 2.8 mm and 4 mm or less, above 4 mm and 5.6 mm or less, above 5.6 mm and 8 mm or less, above 8 mm and 11.2 mm or less, above 11.2 mm and 16 mm or less, or above 16 mm and 22.4 mm or less measured by a method provided in, for example, JIS Z 8815-1994 [Test sieving—General requirements], handling at the time of treating ammonia becomes easy. In addition, the shape of the carrier is not particularly limited, and may be any of e.g. globe, cube and fiber shapes or irregular particles.

As the specific surface area of a carrier increases, the number of nitrifying bacteria which can be supported thereto increases, and therefore the specific surface area is desirably 3.0 m²/g or more, more preferably 4.0 m²/g or more, further desirably 5.0 m²/g or more, yet further preferably 10 m²/g or more, particularly preferably 20 m²/g or more and most preferably 40 m²/g or more. In addition, the upper limit of the specific surface area of a carrier is not particularly limited, and can be preferably 150 m²/g or less, more preferably 100 m²/g or less, further preferably 80 m²/g or less and more suitably 60 m²/g or less. The specific surface area is measured by mercury porosimetry herein.

When using a porous body as a carrier, in order to retain a moderate water retention force and air permeability, the pore volume thereof is preferably 0.6 cm³/g or more, more preferably 0.8 cm³/g or more, further preferably 1.0 cm³/g or more, yet further preferably 1.2 cm³/g or more, particularly preferably 1.4 cm³/g or more and most preferably 1.6 cm³/g or more. On the other hand, a too large pore volume increases the proportion of voids in the porous body, which is a risk that durability will be reduced, and thus the upper limit can be, for example, preferably 4.0 cm³/g or less, more preferably 3.5 cm³/g or less, further preferably 3.0 cm³/g or less and more suitably 2.5 cm³/g or less. The pore volume is measured by mercury porosimetry herein.

The nitrifying bacteria-carrying body in the present invention can be also prepared, for example, by adding activated sludge to a carrier as described above and culturing bacteria on the carrier as desired. In this case, the amount of activated sludge added with respect to the volume of the carrier is a proportion of 5 to 500 vol % such as 5 vol %, 10 vol %, 20 vol %, 30 vol %, 50 vol %, 70 vol %, 100 vol %, 150 vol %, 200 vol %, 300 vol %, 400 vol % and 500 vol %. It should be noted that when the supported nitrifying bacteria are brought into contact with a subject to be treated to initiate an ammonia decomposition reaction, the number of bacteria is changed, and thus the initial amount of seed bacteria inoculated is not a very important parameter. Herein, the contact of a subject to be treated containing an ammonia component and a nitrifying bacteria-carrying body can be performed, for example, by blowing an ammonia-containing gas or adding an ammonia-containing aqueous solution to circulating water.

### <Nitrogen Recovery Method>

The nitrifying bacteria formulation in the present invention can efficiently nitrify ammonia, for example, even under a treatment condition having a high ion concentration, and obtain high-concentration nitrate nitrogen. Therefore, it is suitably used in a method for recovering a nitrogen component in an ammonia component, for example, a nitrogen component in an ammonia-containing gas and an ammonia-containing aqueous solution. The present invention also encompasses a nitrogen recovery method, including a nitrate ion recovery step of decomposing an ammonia component by nitrifying bacteria and recovering a nitrogen component in an ammonia component as nitrate ion, wherein the nitrate ion recovery step includes a wetting step of retaining a nitrifying bacteria formulation in a wet state by supplying circulating water into a microbial decomposition tank retaining the nitrifying bacteria formulation, a contact step of bringing a subject to be treated containing the ammonia component into contact with the nitrifying bacteria formulation in a wet state under an oxygen atmosphere, a decomposition step of decomposing the ammonia component while dissolving the ammonia component and a decomposition product of the ammonia component obtained by decomposition using nitrifying bacteria in the circulating water to accumulate the ammonia decomposition product in the circulating water, and a recovery step of recovering part of or all the circulating water when the concentration of nitrate ion as the ammonia decomposition product in the circulating water is increased to 30,000 mg/L or more to reach a predetermined concentration, wherein the nitrifying bacteria formulation contains ammonia-oxidizing bacteria and nitrite-oxidizing bacteria, the ammonia-oxidizing bacteria contain bacteria belonging to the genus Nitrosococcus, the proportion of the number of the bacteria belonging to the genus Nitrosococcus with respect to the total number of bacteria in the nitrifying bacteria formulation is 0.1% or more, and the proportion of the number of the nitrite-oxidizing bacteria with respect to the total number of bacteria in the nitrifying bacteria formulation is 0.1% or more.

In the nitrogen recovery method in the present invention, water after the treatment in which the decomposition product of the ammonia component has been dissolved is supplied into the microbial decomposition tank as circulating water, and thus nitrate ion, the decomposition product, can be accumulated in the circulating water at a high concentration, for example, a concentration such as 30,000 mg/L or more, or desirably 40,000 mg/L or more, 50,000 mg/L or more, 60,000 mg/L or more, furthermore above 1 M, 70,000 mg/L or more or 100,000 mg/L or more.

In addition, when nitrate ion is accumulated in the circulating water as described above to reach a predetermined concentration, the nitrogen component in the ammonia component can be recovered as a high concentration nitrate ion solution by recovering part of or all the circulating water. Herein, the predetermined concentration is a desired concentration optionally set depending on purposes, and can be, for example, 30,000 mg/L or more, 40,000 mg/L or more, 50,000 mg/L or more, 60,000 mg/L or more, furthermore above 1 M, 70,000 mg/L or more or 100,000 mg/L or more in the present invention.

### (Nitrogen Recovery Device)

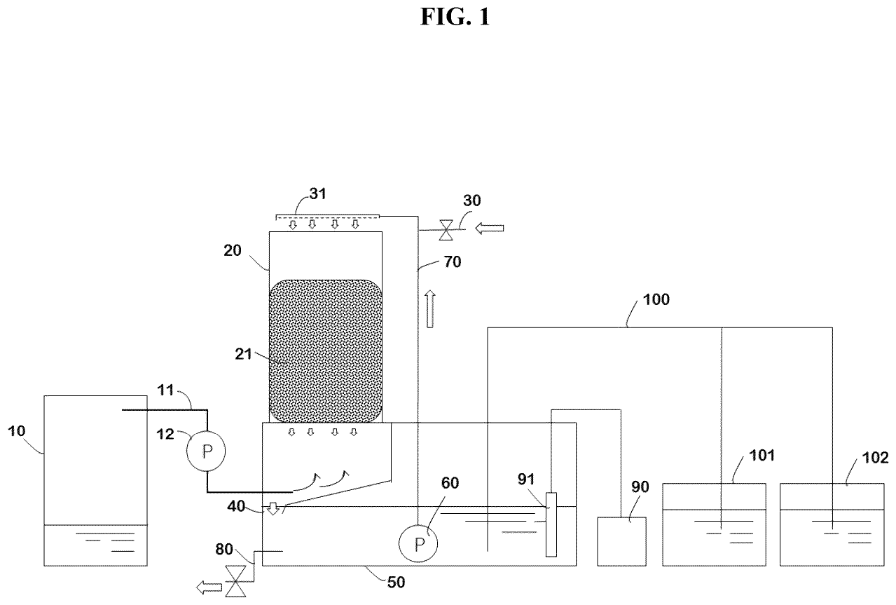

The nitrogen recovery in accordance with the method in the present invention as described above can be performed, for example, using a nitrogen recovery device shown in the description and FIG. 1 according to WO2021/049603 suggested and filed by the present inventors. More particularly, a nitrogen recovery device in an embodiment as described below can be used:
a nitrogen recovery device, including a nitrifying bacteria-carrying body (carrier) containing the nitrifying bacteria formulation as described above, wherein an ammonia component in an ammonia-containing gas is decomposed by nitrifying bacteria, and a nitrogen component in the ammonia component is recovered as an ammonia decomposition product, and including:
(A) a microbial decomposition tank, which has the nitrifying bacteria-carrying body and decomposes the ammonia component under an oxygen atmosphere,
(B) an ammonia-containing gas supplying unit, which supplies the ammonia-containing gas into the microbial decomposition tank,
(C) a water supply unit, which supplies water into the microbial decomposition tank,
(D) a drainage line, which discharges water including the ammonia decomposition product generated in the microbial decomposition tank from the microbial decomposition tank,
(E) a circulating water storage tank, which temporarily stores the water including the ammonia decomposition product discharged from the drainage line,
(F) a retreating line, which connects the storage tank and the microbial decomposition tank and sends the water including the ammonia decomposition product from the storage tank to the microbial decomposition tank,
(G) a circulation unit, which circulates the water including the ammonia decomposition product among the microbial decomposition tank, the drainage line, the circulating water storage tank and the retreating line, and
(H) a recovery unit, which recovers part of or all the water including the ammonia decomposition product having a predetermined nitrate ion concentration from the circulating water storage tank.

For example, using the nitrogen recovery device in the above embodiment, including the nitrifying bacteria-carrying body containing the nitrifying bacteria formulation as described above, a nitrogen component in an ammonia component can be recovered as nitrate ion at a high concentration by supplying an ammonia-containing gas. Herein, both the proportion of existing Nitrosococcus and the existence proportion of NOB in the nitrogen recovery device are preferably 0.1% or more also at sites other than the nitrifying bacteria formulation. When the proportions of the number of the bacteria belonging to the genus Nitrosococcus and the number of nitrite-oxidizing bacteria, for example, in a microbial decomposition tank, particularly in a nitrifying bacteria-carrying body with respect to the total number of bacteria in the microbial decomposition tank or the nitrifying bacteria-carrying body are 0.1% or more, nitrogen recovery can be performed at higher efficiency.

It should be noted that the existence proportions of Nitrosococcus and NOB may be measured by collecting, for example, a part of the nitrifying bacteria-carrying body (as a specific example, the surface portion placed opposite to the surface portion to which an ammonia-containing gas is supplied, of the nitrifying bacteria-carrying body placed in the upper part of the microbial decomposition tank or the nitrifying bacteria-carrying body existing in the microbial decomposition tank), or may be measured by collecting water from a site other than the microbial decomposition tank, for example, a circulating water storage tank. When collecting a part of the nitrifying bacteria-carrying body, it is preferred to avoid collecting it from a site which is not in a wet state.

### (Nitrogen Recovery Conditions)

In the nitrogen recovery method in the present invention, conditions such as the temperature of circulating water are not particularly limited. However, in order to raise the activity of the nitrifying bacteria formulation in the present invention, water temperature is preferably adjusted to, for example, 10 to 60° C., and further suitably 15 to 50° C., particularly 20 to 40° C.

In addition, ammonia nitrification by Nitrosococcus and NOB is an aerobic reaction, and thus it is preferred that oxygen be supplied, for example, by aerating circulating water. The aeration amount is preferably 0.5 L/min or more per L of circulating water, more preferably 1 L/min or more, particularly preferably 3.0 L/min or more and most preferably 4.0 L/min or more. On the other hand, excessive aeration has a risk that vaporization of ammonia will be promoted, and thus the upper limit is preferably 10 L/min or less per L of circulating water, more preferably 8.0 L/min or less and further preferably 6.0 L/min or less.

In the nitrogen recovery method in the present invention, the pH of circulating water is preferably 4.0 to 9.0, more preferably 4.5 to 8.5, further preferably 5.0 to 8.0 and particularly preferably 6.5 to 7.5 from the same reason as in the setting of temperature. The bacteria belonging to the genus Nitrosococcus are AOB relatively resistant to acid, and thus can nitrify ammonia even under such conditions. When the pH of circulating water is about 5.0 or higher, particularly about 6.5 or higher, there is an advantage that residual ammonia is significantly suppressed. When pH is above 9.0, there is a risk that the nitrifying activity will be reduced, and there is also a risk that ammonia is volatilized and leaked.

It should be noted that pH adjustment at the time of nitrogen recovery and culturing may be performed by adding an alkali component such as ammonia gas, ammonia water or an aqueous solution of sodium hydroxide from the outside, and also by allowing a poorly water-soluble weak alkali substance to coexist in a system. The weak alkali substance such as calcium carbonate, magnesium carbonate, dolomite or calcium phosphate is hardly dissolved in neutral water but dissolved under an acid condition, and thus it is dissolved when the surrounding portion is acidified with the progression of the nitrification reaction. Therefore, the pH can be maintained at around neutral. The weak alkali substance is particularly preferably calcium carbonate, and in addition to light calcium carbonate which has been widely used, e.g. heavy calcium carbonate, coral sand and eggshell powder can be used. These weak alkali substances may be added to a microbial decomposition tank and a circulating water storage tank, for example, by incorporating them into a nitrifying bacteria-carrying body, or particles are packed in a dedicated cartridge, which may be connected in series to e.g. a microbial decomposition tank.

In the nitrogen recovery method in the present invention, when ammonia nitrification proceeds to some extent and, for example, the nitrate ion concentration of circulating water reaches a predetermined concentration, 30,000 mg/L or more, the nitrogen content in ammonia can be recovered as nitrate nitrogen by recovering part of or all the water.

Herein, nitrifying bacteria in the nitrifying bacteria formulation can effectively act even after recovering circulating water, and thus when new circulating water is supplied into a system and brought into contact with a subject to be treated containing an ammonia component such as an ammonia-containing gas or an ammonia-containing aqueous solution to restart the decomposition treatment of ammonia, the decomposition reaction of the ammonia component proceeds efficiently as in the case of the previous treatment. Therefore, nitrogen can be repeatedly recovered from the ammonia component without exchanging the nitrifying bacteria formulation. The new circulating water may be e.g. water or activated sludge containing nitrifying bacteria.

In addition, circulating water which is recovered and exchanged at a time may be part of or all (100%) the total amount of circulating water. However, the activity of the nitrifying bacteria can be reduced by suddenly changing environment conditions including circulating water, and thus circulating water which is recovered and exchanged at a time is preferably less than 100% of the total amount of circulating water, more preferably 80% or less and particularly preferably 60% or less. On the other hand, for effective recovery and exchange, it is desirably at least 20% or more of the total amount of circulating water, particularly 30% or more.

In the nitrogen recovery method in the present invention, the proportion of the number of the bacteria belonging to the genus Nitrosococcus with respect to the total number of bacteria in the nitrifying bacteria formulation is 0.1% or more, preferably 1.0% or more, more preferably 3.0% or more, further preferably 7.0% or more and further suitably 10.0% or more. In addition, the proportion of the number of NOB with respect to the total number of bacteria in the nitrifying bacteria formulation is 0.1% or more, preferably 0.5% or more, more preferably 1.0% or more, further preferably 1.5% or more and particularly preferably 2.0% or more. When the rates of the bacteria belonging to the genus Nitrosococcus and NOB are high, ammonia nitrification can be allowed to smoothly proceed even in an environment having a high ion concentration.

It should be noted that the numbers of the bacteria belonging to the genus Nitrosococcus and NOB as described herein are the proportion of the number of bacteria in the nitrifying bacteria formulation at any time point of nitrogen recovery. In the nitrogen recovery method in the present invention, the number of bacteria is changed with the progression of the decomposition step of an ammonia component, and thus the initial number of bacteria and the proportion thereof are not very important parameters. The proportions of the numbers of the bacteria belonging to the genus Nitrosococcus and NOB with respect to the total number of bacteria are only required to be 0.1% or more at any time point of the decomposition step.

However, in order to recover high-concentration nitrate ion, specifically a concentration of 30,000 mg/L or more, particularly 35,000 mg/L or more under a high ion concentration, both the proportion of existing Nitrosococcus and the proportion of existing NOB are desirably 0.1% or more not only in the nitrifying bacteria formulation but also in a subject to be treated (for example, a circulating water storage tank in a treatment device, preferably in a microbial decomposition tank, particularly the upper part of the nitrifying bacteria-carrying body). In addition, in order to recover high-concentration nitrate ion quickly, the proportion of each number of the bacteria belonging to the genus Nitrosococcus and NOB with respect to the total number of bacteria in a subject to be treated is preferably 0.1% or more from the start point of the nitrogen recovery.

In addition, for example, when using pellets of nitrifying bacteria, the amount thereof used is preferably 0.3 mL or more as the volume of pellets obtained by collecting the bacteria belonging to the genus Nitrosococcus with respect to 1 g of the mass of the nitrogen content in ammonia treated for a day, further preferably 0.5 mL or more, especially preferably 1.0 mL or more and particularly preferably 3.0 mL or more; preferably 0.05 mL or more as the volume of pellets obtained by collecting NOB, further preferably 0.1 mL or more, especially preferably 0.5 mL or more and particularly preferably 1.0 mL or more. With the above amounts, ammonia nitrification proceeds more smoothly.

In the nitrogen recovery method in the present invention, the above nitrifying bacteria formulation in the present invention is used, and thus ammonia nitrification proceeds even if the ion (salt) concentration of circulating water is high. Specifically, when the ion concentration in circulating water is 0.5 mol/L or more or the salt concentration is 3 w/v % or more, for example, also when sea water is used as circulating water, ammonia can be nitrified. In addition, even when the treatment of an ammonia component is continued and the nitrate ion concentration in circulating water is increased, the nitrification reaction is not inhibited. Therefore, according to the nitrogen recovery method in the present invention, nitrogen in ammonia can be efficiently recovered as high-concentration nitrate nitrogen without being influenced by the kind of a subject to be treated (e.g. ammonia-containing gas and ammonia-containing aqueous solution), the initial concentration and the number of circulation of water to be treated such as circulating water.

In the nitrifying bacteria formulation in the present invention, when water to be treated contains certain kinds of ion and element in trace amounts, the nitrification ability can be further improved. When water to be treated such as circulating water at the time of nitrogen recovery contains components including elements such as magnesium, phosphorus, potassium and sulfur, the growth of the bacteria belonging to the genus Nitrosococcus and NOB is promoted, and the activity thereof can be also increased. In particular, when magnesium and phosphate ions are included in water to be treated at a concentration equal to or higher than a certain level of concentration, ammonia can be treated more efficiently. It is preferred that in circulating water, the magnesium concentration be 0.5 mM (mmol/L) or more, particularly about 0.6 to 1.0 mM, and the phosphorus concentration be 0.5 mM or more, particularly about 0.6 to 1.0 mM. When the above circulating water, for example, includes a magnesium salt such as MgSO₄·7H₂O at 125 mg/L or more, particularly about 150 to 250 mg/L and a phosphate such as K₂HPO₄ at about 85 mg/L or more, particularly about 100 to 175 mg/L, nitrogen in ammonia can be further efficiently recovered as high-concentration nitrate nitrogen.

### EXAMPLES

The present invention will now be described in more detail by way of examples.

### Examples 1 to 3, Comparative Examples 1 to 3

The effects of the composition of various bacteria in the nitrifying bacteria formulation (the proportion of the number of bacteria) and the salt concentration in a liquid at the time of the nitrification reaction on ammonia nitrification were considered.

Decomposition and nitrification treatments were performed by adding to an ammonia-containing aqueous solution each nitrifying bacteria formulation in a pellet form having each bacterial composition (pellets obtained by solidifying a liquid culture and containing respective bacteria in proportions described in Table 1; hereinafter may be described as “pellet”). The ammonia-containing aqueous solution to which the nitrifying bacteria formulation had been added was put in a container with coral sand to stabilize pH (when the nitrification reaction proceeds and pH becomes acid, because calcium carbonate, the main component of coral sand, is dissolved and neutralized, pH is retained at around neutral), and a silicon stopper with air permeability was put in the container to obtain an aerobic condition. Next, the container was shaken for 5 days while adjusting temperature so that the above ammonia-containing aqueous solution would be exposed to air in the container. The container was shaken using a water bath shaker NTS-1300 manufactured by EYELA.

Specific test conditions are as follows:
Amount of nitrifying bacteria formulation added: 0.1 mL of pellets (2.5 vol %) with respect to 4 mL of an aqueous liquid,
Aqueous liquid (subject to be treated): fresh water including phosphorus, potassium, magnesium and sulfur as trace elements (140 mg/L K₂HPO₄ and 200 mg/L MgSO₄·7H₂O), sea water or a 0.5 M aqueous solution of sodium nitrate: 4 mL,
Ammonia concentration (in terms of the mass of nitrogen atom) in the aqueous liquid (subject to be treated):

In the case of fresh water, 690 mg/L=2.8 mg/4 mL, 927 mg/L=3.7 mg/4 mL in sea water, and 874 mg/L=3.5 mg/4 mL in a 0.5 M aqueous solution of sodium nitrate,
pH: 7.0 (before reaction)
Temperature: 30° C.

It was observed that at least AOB such as the genus Nitrosococcus and the genus Nitrosomonas, and the bacteria belonging to the genus Nitrococcus were included in each activated sludge.

It should be noted that the kind and number of bacteria (the proportion of the number of bacteria) in each activated sludge were analyzed by 16S rRNA bacterial flora analysis using real-time PCR, using primer-probe sets described in Table 2 to Table 4 below. It should be noted that these primers are designed to detect specific bacteria or all bacteria belonging to a specific genus, for example, all bacteria of the genus Nitrosococcus. Both the primer-probe set used in the bacterial flora analysis of the genus Nitrosococcus described in Table 2, and the primer-probe set used in the bacterial flora analysis of the genus Nitrococcus described in Table 3 were designed by the present inventors. In addition, the primer-probe set used in the analysis of AOB such as the genus Nitrosomonas described in Table 4 is those which are conventionally known.

Water to be treated which had been allowed to react with ammonia by shaking for 5 days was collected, and the ion concentrations of ammonium ion, nitrite ion and nitrate ion in the liquid were measured by colorimetry. The degree of ammonia nitrification was evaluated in accordance with criteria described below based on changes in these ion concentrations. The evaluation results are shown with the composition of various bacteria in a nitrifying bacteria formulation in Table 1 below.

### [Evaluation Criteria for the Degree of Ammonia Nitrification]

Evaluation Criteria for Ammonium Ion (NH₄⁺):
Excellent: the ammonium ion concentration in water to be treated was less than 250 mg/L,
Good: the ammonium ion concentration in water to be treated was 250 mg/L or more and less than 500 mg/L,
Fair: the ammonium ion concentration in water to be treated was 500 mg/L or more and less than 750 mg/L, and
Poor: the ammonium ion concentration in water to be treated was 750 mg/L or more.

Evaluation Criteria for Nitrite Ion (NO₂⁻)
Excellent: 90% or more of nitrite nitrogen generated by the oxidation of ammonia in water to be treated was changed to nitrate nitrogen, and the proportion of residual nitrite nitrogen was 10% or less,
Good: 80% or more and less than 90% of nitrite nitrogen generated by the oxidation of ammonia in water to be treated was changed to nitrate nitrogen, and the proportion of residual nitrite nitrogen was above 10% and 20% or less,
Fair: 5% or more and less than 80% of nitrite nitrogen generated by the oxidation of ammonia in water to be treated was changed to nitrate nitrogen, and the proportion of residual nitrite nitrogen was above 20% and 95% or less, and
Poor: only less than 5% of nitrite nitrogen generated by the oxidation of ammonia in water to be treated was changed to nitrate nitrogen, and the proportion of residual nitrite nitrogen was above 95%.

Evaluation Criteria for Nitrate Ion (NO₃⁻)
Excellent: the nitrate ion concentration in water to be treated was increased by 1400 mg/L or more from the start of the test,
Good: the nitrate ion concentration in water to be treated was increased by 700 mg/L or more and less than 1400 mg/L from the start of the test,
Fair: the nitrate ion concentration in water to be treated was increased by 50 mg/L or more and less than 700 mg/L from the start of the test, and
Poor: the nitrate ion concentration in water to be treated was the same as at the start of the test or was increased by less than 50 mg/mL from the start of the test.

TABLE 1
[the proportion of the number of respective bacteria in a nitrifying
bacteria formulation and the results of nitrification test]

| | | | Examples | | | | | |
|---|---|---|---|---|---|---|---|---|
| | | | | | | Comparative | Comparative | Comparative |
| | | | Example 1 | Example 2 | Example 3 | Example 1 | Example 2 | Example 3 |
| Nitrifying bacteria | | | 1 | 2 | 3 | 4 | 5 | 6 |
| formulation No. | | | | | | | | |
| Pro- *¹ | Nitrosococcus | | 0.1 | 4.2 | 11.2 | ND ^{#} | ND ^{#} | ND ^{#} |
| portion | AOB such as | | 2.1 | 2.1 | 0.6 | 5.6 | 2.7 | 1.3 |
| | Nitrosomonas | | | | | | | |
| | Nitrococcus | | 0.1 | 0.9 | 3.9 | <0.1 | ND ^{#} | ND ^{#} |
| Re- *² | In fresh | NH₄⁺ | Excellent | Excellent | Excellent | Good | Good | Good |
| sults of | water | NO₂⁻ | Fair | Good | Excellent | Poor | Poor | Poor |
| nitrify- | | NO₃⁻ | Fair | Good | Excellent | Poor | Poor | Poor |
| cation | In sea | NH₄⁺ | Fair | Good | Excellent | Poor | Poor | Poor |
| test | water | NO₂⁻ | Fair | Good | Excellent | Poor | Poor | Poor |
| | | NO₃⁻ | Fair | Good | Excellent | Poor | Poor | Poor |
| | In *³ | NH₄⁺ | Good | Good | Excellent | Poor | Poor | Poor |
| | Nitrate | NO₂⁻ | Fair | Good | Excellent | Poor | Poor | Poor |
| | | NO₃⁻ | Fair | Good | Excellent | Poor | Poor | Poor |

*¹ The proportion of the number of respective bacteria at the start of the test (%)
*² Evaluation results on each ionic concentration
*³ In an aqueous solution of sodium nitrate (0.5M)
^{#} ND: Not detected

TABLE 2
the primer-probe set used in the bacterial flora
analysis of the genus Nitrosococcus]

| | |
|---|---|
| Forward primer | GTTGACGTTACCYACAGAAGAAGCACC |
| | (SEQ ID NO: 1) |
| Reverse primer | GGGCTTTCACACCCAACTTA |
| | (SEQ ID NO: 2) |
| Probe | AAACCGCCTACGCGCCCTTTACG |
| | (SEQ ID NO: 3) |

TABLE 3
the primer set used in the bacterial flora
analysis of the genus Nitrococcus]

| | |
|---|---|
| Forward primer | CGGGAGGAAAAGCATCGGG |
| | (SEQ ID NO: 4) |
| Reverse primer | TCCGACCGCCTACATGCGC |
| | (SEQ ID NO: 5) |

TABLE 4
the primer set used in the analysis of AOB such
as the genus Nitrosomonas]

| | | |
|---|---|---|
| Forward | CTO 189fA/B | GGAGRAAAGCAGGGGATCG (SEQ ID NO: 6) |
| primer* | CTO 189fC | GGAGGAAAGTAGGGGATCG (SEQ ID NO: 7) |
| Reverse | RT1r | CGTCCTCTCAGACCARCTACTG |
| primer | | (SEQ ID NO: 8) |

*Two kinds of primer were mixed at a proportion of 2:1

It was found that in Examples 1 to 3, using the nitrifying bacteria formulations Nos. 1 to 3 which include the bacteria belonging to the genus Nitrosococcus at 0.1% or more and NOB (in all the above Examples 1 to 3, the bacteria belonging to the genus Nitrococcus) at 0.1% or more with respect to all bacteria in accordance with the present invention, the nitrification reaction proceeded in fresh water, sea water and the like, and the nitrate ion concentration was increased remarkably. In particular, in Example 2 and Example 3, using the nitrifying bacteria formulations No. 2 and 3 in which the proportion of the number of the bacteria belonging to the genus Nitrosococcus was 4.2% or more, especially in Example 3, using the nitrifying bacteria formulation No. 3 (the bacteria belonging to the genus Nitrosococcus at 11.2%), nitrification proceeded remarkably.

On the other hand, in Comparative Examples 1 to 3, using the nitrifying bacteria formulations Nos. 4 to 6 in which the bacteria belonging to the genus Nitrosococcus were not detected, nitrification hardly proceeded in sea water and in an aqueous solution of sodium nitrate having a high concentration, and the ammonium ion concentration in water to be treated was 750 mg/L or more. In fresh water, the nitritation of an ammonia component proceeded by the function of AOB such as Nitrosomonas, but the oxidation of nitrous acid hardly proceeded, and an increase in the nitrate ion concentration in water to be treated was less than 50 mg/L.

It was shown that in order to change nitrogen in ammonia to high-concentration nitrate ion under a condition having a high salt concentration and recover the nitrate ion, the nitrifying bacteria formulation was required to include 0.1% or more of the bacteria belonging to the genus Nitrosococcus and 0.1% or more of NOB. It was also clarified that when the proportion of the bacteria belonging to the genus Nitrosococcus with respect to the total number of bacteria was 1.0% or more (e.g. Example 2 and Example 3), particularly 7.0% or more (e.g. Example 3), ammonia nitrification smoothly proceeded even under a condition having a high salt concentration.

### Example 4, Reference Example

In Example 4 and Reference Example, the nitrification reaction of an ammonia-containing gas was performed using a nitrifying bacteria formulation in a nitrogen recovery device. At this time, the nitrification ability under a treatment condition having a high ion concentration was evaluated by following changes in the concentration of e.g. nitrate ion and changes in the rate of respective bacteria between a case where activated sludge was directly used as seed bacteria in the nitrogen recovery device and a case where a nitrifying bacteria formulation was added to activated sludge.

Specifically, a nitrogen recovery device described in the description according to WO2021/049603, which has a structure as shown in FIG. 1, and has a microbial decomposition tank 20 and an ammonia-containing gas supply line 11, was used. A nitrifying bacteria-carrying body 21, which supports the nitrifying bacteria formulation, was retained in the microbial decomposition tank 20, and water was circulated in e.g. the microbial decomposition tank 20 while blowing an ammonia-containing gas under an oxygen atmosphere. Circulating water and the nitrifying bacteria-carrying body 21 were collected every several weeks to analyze the concentrations of ammonium ion, nitrite ion and nitrate ion in circulating water, and the proportion of respective bacteria (the proportion of the number of bacteria) with respect to all bacteria on the nitrifying bacteria-carrying body 21. The analysis of the proportion of the number of bacteria and the measurement of each ion concentration were performed in the same manner as in Examples 1 to 3. The test results are shown in Table 5 below.

It should be noted that seed bacteria were prepared by adding pellets of a nitrifying bacteria formulation to activated sludge obtained from an activated sludge tank in a pig farm in Saitama prefecture (Example 4). The proportion of added pellets was about 3% of the volume of pellets obtained by collecting bacteria from the same amount of activated sludge. Seed bacteria after the addition of the pellets were supported on a porous glass (hereinafter may be referred to as “nitrifying bacteria-carrying body”). Supporting seed bacteria on the carrier was performed by inoculating 3 L (corresponding to a proportion of 300 vol % of the packed volume of the porous glass) of seed bacteria to 340 g (corresponding to 1 L of the packed volume) of the porous glass. For comparison, the nitrification treatment was performed using activated sludge directly supported thereto without adding pellets (Reference Example).

The decomposition treatment of an ammonia-containing gas in the nitrogen recovery device was performed in conditions below:
Nitrifying bacteria-carrying body 21

Packed height: 20 cm (packed volume: 1 L), temperature: 30° C., water content (in the microbial decomposition tank 20): 13 vol %,
Nitrifying bacteria formulation: containing 10.59% of Nitrosococcus, 0.14% of AOB such as Nitrosomonas and 1.85% of Nitrococcus (in all the bacteria, the proportion of the number of bacteria with respect to the total number of bacteria in the nitrifying bacteria formulation),
Circulating water: 3 L of activated sludge was directly used,

Temperature: 30° C., pH: 7.0 or higher (when pH is lower than 7.0, pH is automatically adjusted by adding a 0.5 N aqueous solution of sodium hydroxide by a pH controller),

Liquid amount: 3 times the volume of the nitrifying bacteria-carrying body 21,

Circulating amount per hour: 400 mL/min per L of the packed volume of the nitrifying bacteria-carrying body 21, and aeration amount in circulating water: 4 L/min with respect to 3 L of circulating water, and
Amount of added ammonia

about 250 mg/day in terms of the mass of nitrogen atom.

TABLE 5
[the proportion of the number of respective bacteria
and each ion concentration in circulating water]

| | | Example 4 | | Reference Example | |
|---|---|---|---|---|---|
| Elapsed days | | Initial | Day 111 | Initial | Day 111 |
| Proportion of | Nitrosococcus | 0.31 | 1.21 | ND^{#} | ND^{#} |
| number of | Other AOB (**) | 1.54 | 3.95 | 1.54 | 14.75 |
| bacteria * ¹ | Nitrococcus | 0.07 | 1.81 | ND^{#} | ND^{#} |
| Each ion | NH₄⁺ | 61 | 1059 | 62 | 2073 |
| concentration * ² | NO₂⁻ | 539 | 86 | 664 | 12862 |
| | NO₃⁻ | 1329 | 51114 | 772 | 3923 |
| Rate of nitrogen | N (NH₄⁺) | 9.2 | 6.6 | 11.4 | 24.2 |
| form * ³ | N (NO₂⁻) | 32.1 | 0.2 | 47.6 | 58.9 |
| | N (NO₃⁻) | 58.7 | 93.1 | 41.0 | 16.9 |

* ¹ The proportion of the number of cells to all bacterial cells in the microorganism decomposition tank (%)
* ² The concentration (mg/L) in terms of the mass of nitrogen atom
* ³ The rate of each nitrogen form to the total nitrogen concentration of ions (%)
(**) e.g., Nitrosomonas
^{#}ND: Not detected

In Example 4 to which a nitrifying bacteria formulation including 0.1% or more of the bacteria belonging to the genus Nitrosococcus and 0.1% or more of NOB (Nitrococcus) with respect to all bacteria in accordance with the present invention was added, the existence proportions of the bacteria belonging to the genus Nitrosococcus and Nitrococcus were above 1%, and the nitrate ion concentration was increased to above 50,000 mg/L after 111 days. In Example 4, ammonia nitrification could be allowed to proceed even under an environment having a high ion concentration such as a nitrate ion concentration of near 50,000 mg/L. On the other hand, in Reference Example to which a nitrifying bacteria formulation was not added, nitrous acid was accumulated at a high concentration of above 10,000 mg/L. In addition, although the bacteria of the genus Nitrosomonas were detected in a large amount, the ammonium ion concentration was increased to near twice the concentration in Example 4, and the nitrate ion concentration was remarkably low compared to that in Example 4 after 111 days.

### Example 5

The same device as in Example 4 was used except that a 200 L water tank (used as a foam glass packed tank) and a 100 L water tank (used as a circulating water tank) were provided, and culturing a nitrifying bacteria formulation was tried. In the 200 L water tank, 10 L of a foam glass to which the bacteria belonging to Nitrosococcus and the bacteria belonging to Nitrococcus had been supported (proportion of existing Nitrosococcus: 6.4% and proportion of existing Nitrococcus: 4.2%) was put together with 190 L of a foam glass to which the bacteria had not been supported, and the same test as in Example 4 was performed. However, 50 L of artificial sea water having a composition described below was used as circulating water (medium), and the device was operated for 51 days in operating conditions below. In addition, ammonia was injected in the form of not gas but an aqueous solution. The amount of added ammonia per hour was 206 mg as the initial value, and the next added amount was set to increase to 1.2 to 1.5 times the above amount when the pH of circulating water was lower than 7 after 30 minutes from addition.

### <Composition of Circulating Water>

Artificial sea water salt (Sea Water manufactured by GEX CORPORATION, salt concentration: 36 g/L (about 0.6 M)),

Amount of added K₂HPO₄: 114 mg/L,

Amount of added MgSO₄·7H₂O: 200 mg/L.

### <Operating Conditions>

Water temperature: 30° C.,

Amount of circulating water: 30 L/min, and

Aeration: 60 L/min per 50 L of circulating water.

The proportion of existing Nitrosococcus and the proportion of existing Nitrococcus were increased to 24.5% and 11.3%, respectively, after the operation for 51 days. Consequently, the amount of ammonia (in terms of ammonium ion) which the whole device can nitrify could be increased to 55,170 mg/day at day 51, ten times or more the amount at the start of culturing. It was revealed that in a system to which a nitrifying bacteria formulation including 0.1% or more of the bacteria belonging to the genus Nitrosococcus and 0.1% or more of NOB with respect to all bacteria in accordance with the present invention was added, a large amount of ammonia could be oxidized and decomposed while increasing the number of the bacteria belonging to the genus Nitrosococcus and the bacteria belonging to the genus Nitrococcus by culturing even under a condition having a high salt (ion) concentration.

As described above, the present invention has provided a nitrifying bacteria formulation that is capable of enabling a sufficient nitrification reaction to proceed even under various environments, for example, an environment having a high ion concentration (salt concentration), and efficiently recovering high-concentration nitrate nitrogen from ammonia produced in animal husbandry facilities, composting facilities, sewage treatment plants and the like, and a nitrogen recovery method.

1. A nitrifying bacteria formulation, comprising: ammonia-oxidizing bacteria and nitrite-oxidizing bacteria,
the ammonia-oxidizing bacteria containing bacteria belonging to genus Nitrosococcus,
a proportion of a number of the bacteria belonging to the genus Nitrosococcus with respect to a total number of bacteria in the nitrifying bacteria formulation being 0.1% or more, and
a proportion of a number of the nitrite-oxidizing bacteria with respect to a total number of bacteria in the nitrifying bacteria formulation being 0.1% or more.

2. The nitrifying bacteria formulation according to claim 1, wherein the proportion of the number of the bacteria belonging to the genus Nitrosococcus with respect to the total number of bacteria in the nitrifying bacteria formulation is 1.0% or more.

3. The nitrifying bacteria formulation according to claim 1, wherein the proportion of the number of the bacteria belonging to the genus Nitrosococcus with respect to the total number of bacteria in the nitrifying bacteria formulation is 7.0% or more.

4. The nitrifying bacteria formulation according to claim 1, wherein the nitrite-oxidizing bacteria contain bacteria belonging to genus Nitrococcus.

5. The nitrifying bacteria formulation according to claim 1, further comprising a liquid culture of the ammonia-oxidizing bacteria and the nitrite-oxidizing bacteria.

6. The nitrifying bacteria formulation according to claim 1, further comprising pellets or freeze-dried product of the ammonia-oxidizing bacteria and the nitrite-oxidizing bacteria.

7. The nitrifying bacteria formulation according to claim 1, further comprising a nitrifying bacteria-carrying body in which the ammonia-oxidizing bacteria and the nitrite-oxidizing bacteria are supported on a carrier.

8. A method for culturing the ammonia-oxidizing bacteria and the nitrite-oxidizing bacteria contained in the nitrifying bacteria formulation according to claim 1, the method comprising:
bringing seed bacteria comprising the bacteria belonging to the genus Nitrosococcus and the nitrite-oxidizing bacteria into contact with an ammonia component in a medium in a wet state under an oxygen atmosphere with a salt concentration maintained at 0.5 mol/L or more and 1 mol/L or less.

9. The culturing method according to claim 8, wherein the medium has a magnesium concentration of 0.5 mmol/L or more as well as a phosphorus concentration of 0.5 mmol/L or more.

10. The nitrifying bacteria formulation according to claim 2, wherein the nitrite-oxidizing bacteria contain bacteria belonging to genus Nitrococcus.

11. The nitrifying bacteria formulation according to claim 3, wherein the nitrite-oxidizing bacteria contain bacteria belonging to genus Nitrococcus.

12. The nitrifying bacteria formulation according to claim 2, further comprising a liquid culture of the ammonia-oxidizing bacteria and the nitrite-oxidizing bacteria.

13. The nitrifying bacteria formulation according to claim 3, further comprising a liquid culture of the ammonia-oxidizing bacteria and the nitrite-oxidizing bacteria.

14. The nitrifying bacteria formulation according to claim 4, further comprising a liquid culture of the ammonia-oxidizing bacteria and the nitrite-oxidizing bacteria.

15. The nitrifying bacteria formulation according to claim 2, further comprising pellets or freeze-dried product of the ammonia-oxidizing bacteria and the nitrite-oxidizing bacteria.

16. The nitrifying bacteria formulation according to claim 3, further comprising pellets or freeze-dried product of the ammonia-oxidizing bacteria and the nitrite-oxidizing bacteria.

17. The nitrifying bacteria formulation according to claim 4, further comprising pellets or freeze-dried product of the ammonia-oxidizing bacteria and the nitrite-oxidizing bacteria.

18. The nitrifying bacteria formulation according to claim 2, further comprising a nitrifying bacteria-carrying body in which the ammonia-oxidizing bacteria and the nitrite-oxidizing bacteria are supported on a carrier.

19. The nitrifying bacteria formulation according to claim 3, further comprising a nitrifying bacteria-carrying body in which the ammonia-oxidizing bacteria and the nitrite-oxidizing bacteria are supported on a carrier.

20. The nitrifying bacteria formulation according to claim 4, further comprising a nitrifying bacteria-carrying body in which the ammonia-oxidizing bacteria and the nitrite-oxidizing bacteria are supported on a carrier.
